(19) United States
(12) Patent Application Publication
(10) Pub. No.: US 2004/0101521 A1
(43) Pub. Date: May 27, 2004

(54) Iron sequestration or elimination to reduce neurodegeneration or Parkinsons disease progression

(75) Inventor: Julie K. Andersen, Novato, CA (US)

Correspondence Address: QUINE INTELLECTUAL PROPERTY LAW GROUP, P.C., P O BOX 458, ALAMEDA, CA, 94501, US

(73) Assignee: The Buck Institute for Research on Aging

(21) Appl. No.: 10/618,444

(22) Filed: Jul. 11, 2003

Related U.S. Application Data
(60) Provisional application No. 60/395,691, filed on Jul. 12, 2002.

(51) Int. Cl. A61K 48/00; A61K 31/47; A61K 31/195; A61K 38/40

(52) U.S. Cl. 424/93.21; 514/6; 514/311; 514/566

(57) ABSTRACT

This invention pertains to the discovery that elevated level of free iron causal in the onset and/or progression of diseases characterized by neural degeneration (e.g., Parkinson's Disease). It was also discovered that lowering free iron levels can inhibit (e.g. reduce or eliminate) the onset and/or progression of one or more symptoms of such diseases. Thus, in one embodiment this invention provides a method of inhibiting neural degeneration in a mammal. The method involves reducing free iron levels in a neural tissue of said animal in an amount sufficient to inhibit neural degeneration in said neural tissue.

### CROSS-REFERENCE TO RELATED APPLICATIONS

This application claims benefit of and priority to U.S. Ser. No. 60/395,691, filed on Jul. 12, 2002, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported, in part, by National Institutes of Health Grant Nos: AG12141 and AG41264. The Government of the United Stets of America my have certain rights in this invention.

### FIELD OF THE INVENTION

This invention pertains to the field of neurobiology. In particular, this invention pertains to the discovery that lowering free iron levels can inhibit (reduce or eliminate) the onset or progression of diseases characterized by neural degeneration (e.g., Parkinson's Disease).

### BACKGROUND OF THE INVENTION

Ageing of the population due to increased life expectancy has brought with it a major increase in cognitive disorders associated with normal cerebral ageing and with pathological cerebral ageing occurring in the course of neurodegenerative diseases such as, for example, Alzheimer's disease. Parkinson's disease, and the like.

The majority of substances used today in treating cognitive disorders associated with ageing act by facilitating the central cholinergic systems—either directly, as in the case of acetylcholinesterase inhibitors (tacrine, donepezil) and cholinergic agonists (nefiracetam), or indirectly, as in the case of nootropic agents (piracetam, pramiracetam) and cerebral vasodilators (vinpocetine).

Iron levels in the substantia nigra (SN), the dopamine-containing region of the brain that undergoes selective degeneration in Parkinson's disease (PD), have been reported to be elevated in patients with the disorder (Sofic et al. (1988) J. Neural. Transm. 74: 199-205; Sofic et al. (1991) J. Neurochem. 56: 978-982′ Dexter et al. (1987) Lancet 21: 1219-1220; Dexter et al. (1989) J. Neurochem. 52: 1830-1836; Youdim et al. (1993) Mov. Dis. 8: 1-12; Gerlach et al. (1994) J. Neurochem. 63:793-807; Yantiri and Andersen (1999) IUBMB Life 48: 1-3; Griffiths et al. (1999) Brain 122: 667-673; Andersen, J. K. (2001) Pp. 11-25 In: Ageing Vulnerability: Causes and Interventions, Novartis Foundation Symposium 235, John Wiley and Sons, Inc). Accessible ferrous iron (Fe²⁺) can react with hydrogen peroxide (H₂O₂) produced during oxidative deamination of dopamnine to generate hydroxyl radicals (⁻OH) which can damage proteins, nucleic acids, and membrane phospholipids leading to cellular degeneration (Beal (1992) Ann. Neurol. 31: 119-130; Gutteridge (1992) Ann. Neurol. 32: S16-S21). Whether the increase in SN iron is a causal factor in the disease or a consequence itself of neuronal degeneration has not been determined (Adams and Odunze (1991) Free Radic Biol. Med. 10, 161-169; Berg et al. (2001) J. Neurochem. 79: 225-236; Thompson et al. (2001) Brain Res. Bull. 55: 155-164).

### SUMMARY OF THE INVENTION

This invention pertains to the discovery that elevated level of free iron causal in the onset and/or progression of diseases characterized by neural degeneration (e.g., Parkinson's Disease). Moreover, it was a surprising discovery that lowering free iron levels can inhibit (e.g. reduce or eliminate) the onset and/or progression of one or more symptoms of such diseases. Exploiting this discovery this invention provides methods for inhibiting the onset and/or progression of such diseases.

This invention pertains to the discovery that elevated level of free iron causal in the onset and/or progression of diseases characterized by neural degeneration (e.g., Parkinson's Disease). Moreover, it was a surprising discovery that lowering free iron levels can inhibit (e.g. reduce or eliminate) the onset and/or progression of one or more symptoms of such diseases. Exploiting this discovery this invention provides methods for inhibiting the onset and/or progression of such diseases.

Thus, in one embodiment, this invention provides a method of inhibiting neural degeneration in a mammal. The method typically involves reducing free iron levels in a neural tissue (e.g. brain tissue, nerve of CNS, nerve of peripheral nervous system, etc.) of said animal in an amount sufficient to inhibit neural degeneration in the neural tissue. In certain embodiments, the free iron levels are reduced by binding or chelating the iron by contacting the iron with an agent that binds or chelates iron. Suitabe agents include, but are not limited to small organic molecules (e.g. 5-chloro-7-iodo-8-hydroxyquinoline (clioquinol), deferiprone, desferrioxamine, pseudan, derivatives thereof, etc.) proteins (e.g. ferritin, ferritin heavy subunit (H-ferritin), etc.). The agent(s) can be administered by any of a number of convenient means including, but not limited to systemic administration (e.g. i.v. injection, i.p. injection, inhalation, transdermal delivery, oral delivery, nasal delivery, rectal delivery, etc.) and/or local administration (e.g. direct injection into a target tissue, delivery into a tissue via cannula, delivery into a target tissue by implantation of a time-release material), delivery into a tissue by a pump, etc. Where the agent is a protein, it can be chemically synthesized ex vivo, recombinantly expressed ex vivo, recombinantly expressed in vivo (e.g. using “gene therapy” methods), and the like. In certain embodiments, the protein is recombinantly expressed in a nerve, cell. The mammal can be a human (e.g. a human diagnosed as having or at risk for Parkinson's disease), or a non-human mammal. In certain embodiments, the inhibition of neural degeneration comprises reducing dopaminergic cell loss.

In another embodiment, this invention provides a method of inhibiting the onset or progression of a disease characterized by neural degeneration in a mammal. This method typically involves reducing free iron levels in a neural tissue of a mammal having or at risk for a disease characterized by neural degeneration (e.g. Parkinson's disease). In certain embodiments, the free iron levels are reduced by binding or chelating the iron by contacting the iron with an agent that binds or chelates iron, e.g. using one or more of the materials and/or methods described above.

In still another embodiment, this invention provides a method of mitigating one or more symptoms of a disease (e.g. Parkinson's disease) characterized by neural degeneration in a mammal. The method typically involves administering to the mammal an agent that the sequestration or chelation of free iron in the mammal in an amount to mitigate one or more symptoms of said disease. In certain embodiments, the method involves administering an iron chelator to the mammal and/or administering a construct to the mammal that expresses an iron chelator (e.g. ferritin, H ferritin, hemoglobin, etc.), or induces the upregulation of an endogenous iron chelator (e.g., endogenous ferritin, hemoglobin, etc.). In certain embodiments, the iron-chelator include, but is not limited to clioquinol, deferiprone, desferrioxamine, pseudan, and derivatives thereof. In certain embodiments, the iron-binding protein is recombinantly expressed in vivo (e.g. in a nerve cell or other cell(s) associated with neural tissue). The mammal can be a human (e.g. a human diagnosed as having or at risk for Parkinson's disease) or a non-human mammal. The inhibition of neural degeneration can comprise a reduction of dopaminergic cell loss.

This invention also provides a method of inhibiting the onset or progression of a disease characterized by neural degeneration in a mammal. The method typically involves reducing free iron levels in a neural tissue of a mammal having or at risk for a disease characterized by neural degeneration (e.g. Parkinson's disease, Alzheimer's disease, ALS, etc.). In certain embodiments,-the free iron levels are reduced by binding or chelating the iron by contacting the iron with an agent that binds or chelates said iron. In certain embodiments the agent is an iron-chelating small organic molecule (e.g. clioquinol, deferiprone, desfenioxamine, pseudan, derivatives thereof, etc.). In certain embodiments the agent is an iron-binding protein (e.g. ferritin, H ferritin, hemoglobin, hemoglobin fragments, etc). The protein can be a native (e.g. upregulated endogenous protein), a heterologous protein, a recombinantly expressed protein (e.g. expressed ex vivo or in vivo), and the like. In certain embodiments, the protein is recombinantly expressed in a nerve cell. The mammal can be a human (e.g. a human diagnosed as having or at risk for Parkinson's disease), or a non-human mammal. In certain embodiments, the inhibition of neural degeneration comprises reducing dopaminergic cell loss.

In still another embodiment, this invention provides a kit for mitigating the onset or progression of a disease characterized by neural degeneration in a mammal. The kit typically includes an agent that sequesters and/or chelates free iron in a mammal, and/or a construct that expresses an agent that sequesters and/or chelates free iron in a mammal, and/or an agent that upregulates the expression of an endogenous chelator of free iron in a mammal.; and instructional materials teaching the sequestration or chelation of free iron to mitigate the onset or progression of a disease characterized by neural degeneration in a mammal. In certain embodiments, the agent can be formulated in a unit dosage formulation for mitigating the onset or progression of a disease characterized by neural degeneration in a human (e.g. Parkinson's disease). In certain embodiments, the agent comprises a nucleic acid that encodes a protein that chelates iron (e.g. ferritin, H ferritin, hemoglobin, etc.). In certain embodiments, the agent comprises an iron chelator (e.g. clioquinol, deferiprone, desferrioxamine, pseudan, and derivatives thereof, etc.).

Also provided is a pharmaceutical composition for mitigating the onset or progression of a disease characterized by neural degeneration in a mammal. The composition typically comprises an agent that increases sequestration or chelation of free iron in said mammal. The agent can be an agent that itself increases sequestration or chelation of free iron, an agent/construct that expresses a protein that sequesters and/or chelates free iron, an agent that upregulates endogenous sequesters/chelators of free iron, and the like. The composition can be formulated in a unit dosage formulation for mitigating the onset or progression of a disease characterized by neural degeneration in a human (e.g. Alzheimer's disease, Parkinson's disease, ALS, etc.). In certain embodiments the agent comprises a nucleic acid that encodes a protein that chelates iron. Suitable proteins include, but are not limited to ferritin, ferrition fragments/derivatives (e.g. H ferritin), hemoglobin, hemoglobin fragments/derivatives, and the like.

In certain embodiments, this invention provides a neural tissue (e.g. in a mammal diagnosed as having or at risk for a disease characterized by neural degeneration) in contact with an agent that chelates and/or sequesters free iron. In certain embodiments, the mammal is one not diagnosed as having an iron overload disease. In certain embodimenents, the agent is an iron chelator (e.g. clioquinol, deferiprone, desferrioxamine, pseudan, and derivatives thereof), and/or an iron-binding protein (e.g. ferritin, H ferritin, hemoglobin, etc.). The iron-binding protein can be recombinantly expressed, e.g. as described herein.

This invention also provides a method of evaluating the risk or progression of a disease (e.g. Parkinson's disease, Alzheimer's disease) characterized by neural degeneration in a mammal. The method involves providing a biological sample from the mammal; and determining the level of free iron in the sample where an elevated level of free iron as compared to that found in a sample from a normal healthy mammal indicates that the mammal is at risk for or progressing with said disease.

### Definitions

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes.

### BRIEF DESCRIPTION OF THE DRAWINGS

FIGS. 1A-1D illustrates the creation of pTH-ferritin transgenics. FIG. 1A: Schematic of pTH-ferritin construct used for creation of ferritin transgenics and Xba I-EcoRI probe used for Southern analysis. pTH, 4.8 kb 5′ rat tyrosine hydroxylase promoter region; human ferritin H-chain, human ferritin heavy chain 2.6 kb genomic fragment; SV40/poly A, 900 bp 3′ large T antigen SV40 splice/polyadenylation sequences; probe, 32P-labeled Xba I/EcoRI cDNA fragment of ferritin H-chain. FIG. 1B: Representative Southern blot analysis of genomic tail DNA isolated from pTH-ferritin founders. Lanes 1, 2, transgenics, lanes 3-5, non-transgenics, lane 6, 2.6 kb Xba I/EcoRI ferritin probe. FIG. 1C: Representative Western blot using monoclonal antibody directed against human H-ferritin. Tg, ferritin transgenic; Wt wild-type littermate. Arrow shows expected position of the 21 kD human ferritin H-chain protein. FIG. 1D: Expression of human ferritin protein product in dopaminergic SN neurons verified by double immunocytochemistry (ICC) using H-ferritin and TH antibodies. 1-3, ICC with monoclonal antibody against human H-ferritin (hFh dilution, 1:500). 4-6, ICC with TH antibody (dilution, 1:500). 1 and 4, 10× magnification, 2, 3, 5 and 6, 20× magnification

FIGS. 2A, 2B, and 2C show levels and localization of ferric/ferrous iron in the SN of pTH-ferritin transgenics vs. wild-type littermates. FIG. 2A: Representative MRI analysis of brains from ferritin transgenics vs. wild-type animals demonstrating levels of ferritin-bound ferric iron, n=4 for each group, SN, substantia nigra; wt, wild-type; Tg, transgenic. FIG. 2B: Bioavailable SN ferrous iron levels, n=5 for each group. *p<0.01. FIG. 2C: Localization of SN ferric iron within dopaminergic neurons in the ferritin transgenics as verified by double staining of Perls-positive SN cells with TH antibody. 1, 4× magnification of Perls staining in a representative section of the SN region of a ferritin transgenic mouse; 2, 10× magnification of boxed region, panel 1 highlighting position of a TH+ dopaminergic SN neuron in this brain area (arrow); 3, Perls staining of the dopaminergic neuron highlighted in panel 2; 4, 40× magnification of the dopaminergic neuron shown in panel 2 demonstrating TH positivity.

FIGS. 3A and 3B show the effects of MPTP administration on induction of oxidative stress in pTH-ferritin transgenics vs. wild-type littermates. FIG. 3A: Percentage change in ROS levels in SN tissue 8 hrs following MPTP administration, p<0.01. FIGS. 3A:B: Measurement of percentage change in GSH levels in SN tissue 2 hrs and 8 hrs following MPTP administration, untreated vs. MPTP-treated ferritin transgenics, p>0.05. White bars 2 hours, black bars 8 hours.

FIGS. 4A and 4B show the effects of MPTP administration on dopaminergic SN neuronal cell numbers and striatal (ST) dopamine and its metabolites in pTH-ferritin transgenics vs. wild-type littermates. FIG. 4A: TH+SN cell counts from transgenics vs. non-transgenics 7 days following MPTP administration, saline vs. MPTP-treated WT, p<0.001. FIG. 4B: ST DA content in ferritin transgenic vs. wild-type littermates 7 days following MPTP, saline vs. MPTP-treated WT, p<0.001. C: ST DOPAC and HVA content in ferritin transgenic vs. wild-type littermates, saline vs. MPTP-treated WT, p<0.001.

FIGS. 5A and 5B show the effects of CQ pretreatment on total SN iron content. FIG. 5A: Total SN iron content (μg/mg tissue wet weight) measured via mass spectrometry of saline (Sal) vs. CQ-fed animals, p<0.01. FIG. 5B: SN iron levels measured via MRI in saline-fed vs. CQ-fed animals, p<0.01.

FIGS. 6A, 6B, and 6C illustrate the effects of CQ pretreatment against MPTP-mediated oxidative stress. FIG. 6A: Levels of 4-HNE-protein conjugates and FIG. 6B: protein carbonyl content as assessed by slot blot analysis of SN tissue 24 hrs following MPTP or saline (Sal) administration in the absence or presence of CQ pretreatment, Sal/Sal vs. SalMPTP, *p<0.01; Sal/MPTP vs. CQ/Sal or CQ/MPTP, **p<0.01. FIG. 6C: Total SN GSH levels 24 hrs following MPTP or saline administration ±CQ pretreatment, Sal/Sal vs. Sal/MPTP, *p<0.01; Sal/MPTP vs. CQ/Sal or CQ/MPTP, **p<0.01.

FIGS. 7A and 7B illustrate the protective effects of CQ pretreatment against MPTP-mediated SN dopaminergic cell loss. FIG. 7A: ST dopamine content from CQ vs. vehicle-fed animals 7 days following MPTP or-saline (Sal) administration, Sa/Sal vs. Sal/MPTP, *p<0.01; Sal/MPTP vs. CQ/MPTP, **p<0.01. FIG. 7B: TH⁺SN cell counts from CQ or vehicle-fed animals 7 days following MPTP or saline administration, Sal/Sal vs. Sal/MPTP, *p<0.01; Sal/MPTP vs. CQ/MPTP, **p<0.01.

### DETAILED DESCRIPTION

This invention pertains to the discovery that elevated levels of free iron appear to be implicated in the etiology of the onset or progression of diseases characterized by neural degeneration (e.g., Parkinson's Disease). Indeed, it was a discovery of the present inventor that free iron is causal in the onset and/or progression of such diseases. Moreover, it was a surprising discovery that lowering free iron levels can inhibit (e.g. reduce or eliminate) the onset and/or progression of one or more symptoms of such diseases.

Thus, in various embodiments, this invention contemplates the use of agents that reduce free iron levels in a neural tissue to inhibit neural degeneration (e.g. the loss of dopaminergic neurons) in a mammal. The agents can be used to inhibit the onset and/or progression of the disease or to mitigate one or more symptoms of the disease.

### I. Reduction of Free Iron.

A number of methods can be utilized to reduce free iron levels in the subject organism (e.g. human, non-human mammal). Such methods include, for example, the administration of iron chelators to the subject organism, the expression of iron chelating proteins in the organism, the use of agents that upregulate the production of iron chelating proteins in the organism, and the like.

Iron chelators are well known to those of skill in the art. The binding of chelators to iron reduces or blocks the ion's ability to catalyze redox reactions. Iron ions typically have six electrochemical coordination sites. Consequently, a chelator molecule that binds to all six sites can completely inactivates the “free” iron. Such chelators are termed “hexidentate”, of which desferrioxamine is an example.

With some chelators, a single molecule interactions with only two of-the coordination sites on iron. These chelators are called, “bidentate”. An example of this type of molecule is ferrichrome. Three molecules coordinate with a single iron ion to produce complete chemical immobilzation. Another example is deferiprone, or “L1”.

Hexidentate chelators have the advantage of inactivating iron as part of a 1:1 molecular complex. On the other hand, bidentate chelators can produce partial reaction products with iron (Fe): Fe(C) [redox reactive], FeC2 [redox reactive], and FeC3 [inactive]. With a bidentate iron chelator, a spectrum of chemical species will exist, of which a minority is inactive. I such contexts, a chemical excess of chelator can be used to push the reaction toward completion, the formation of the FeC3 (inactive) product.

Iron chelators are well known to those of skill in the art. Such chelators include, but are not limited to 5-chloro-7-iodo-8-hydroxyquinoline (clioquinol), deferiprone, desferrioxamine, pseudan, and the like. One new class of iron chelators includes the exochelins. The use of exochelins and exochelin variants to chelate free iron is described in detail in U.S. Pat. No. 5,721,209.

Proteins that bind iron are also known to those of skill in the art. Such proteins include, but are not limited to ferritin, hemoglobin, and the like.

Similarly, agents that upregulate endogenous production of such proteins are also known to those of skill in the art.

### II. Modes of Administration.

The mode of administration of the iron chelating agent(s) depends on the nature of the particular agent. Small molecule chelators can be provided in “standard” pharmaceutical formulations. Heterologous nucleic acids encoding various iron binding proteins can be provided in a form suitable for “genetic delivery methods”. Such nucleic acids can be delivered and expressed in target cells (e.g. brain cells) using methods of gene therapy, e.g. as described below.

### A) Pharmaceutical Formulations

The compositions of the invention include bulk drug compositions useful in the manufacture of non-pharmaceutical compositions (e.g., impure or non-sterile compositions) and pharmaceutical compositions (i.e., compositions that are suitable for administration to a subject or patient) that can be used directly and/or in the preparation of unit dosage forms. Such compositions comprise a therapeutically effective amount of one or more therapeutic agents (e.g. iron chelating agent(s)) disclosed herein or a combination of the agent(s) and a pharmaceutically acceptable carrier.

The iron chelating agents used in the methods of this invention, (e.g. to reduce neurological degeneration) can be prepared and administered in a wide variety of rectal, oral and parenteral dosage forms for treating and preventing neurological damage. One or more iron chelating agent(s) can be administered by injection, that is, intravenously, intramuscularly, intracutaneously, subcutaneously, intraduodenally, or intraperitoneally. Also, the compounds can be administered by inhalation, for example, intranasally. Additionally, certain compounds can be administered transdermally.

In a specific embodiment, the term ∘pharmaceutically acceptable∘ means approved by a regulatory agency of the Federal or a state government or listed in the U.S. Pharmacopeia or other generally recognized pharmacopeia for use in animals, and more particularly in humans, or suitable for administration to an animal or human. The term “carrier” refers to a diluent,,adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of the compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Pharmaceutical compositions comprising the iron chelating agents, or upregulators of iron binding protein expression can be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries that facilitate processing of the molecules into preparations that can be used pharmaceutically. Proper formulation is dependent upon the route of administration chosen.

For topical or transdermal administration, the iron chelating agent(s) expression and/or activity can be formulated as solutions, gels, ointments, creams, lotion, emulsion, suspensions, etc. as are well-known in the art. Systemic formulations include those designed for administration by injection, e.g. subcutaneous, intravenous, intramuscular, intrathecal or intraperitoneal injection, as well as those designed for transdermal, transmucosal, inhalation, oral or pulmonary administration.

For injection, the iron chelating agent(s) can be formulated in aqueous solutions, preferably in physiologically compatible buffers such as Hanks's solution, Ringer's solution, or physiological saline buffer. The solution can contain formulatory agents such as suspending, stabilizing and/or dispersing agents. Alternatively, compositions comprising the iron chelating agent(s) can be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the i iron chelating agent(s) can be readily formulated by combining chelating agent(s) with pharmaceutically acceptable carriers well known in the art. Such carriers enable the chelating agent(s) to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, e.g. lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added.

For buccal administration, the iron chelating agent(s) can take the form of tablets, lozenges, etc. formulated in conventional manner.

For administration by inhalation, the iron chelating agent(s) for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator may be formulated containing a powder mix of the iron chelating agent(s) and a suitable powder base such as lactose or starch.

the iron chelating agent(can also be formulated in rectal or vaginal compositions such as suppositories or retention enemas, e.g, containing conventional suppository bases such as cocoa butter or other glycerides.

In addition to the formulations described previously, the iron chelating agent(s) can also be formulated as a depot preparation. Such long acting formulations may be administered by implantation (for example subcutaneously or intramuscularly) or by intramuscular injection. Thus, for example, the iron chelating agent(s) can be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that may be used to deliver the iron chelating agent(s). Certain organic solvents such as dimethylsulfoxide also may be employed, although usually at the cost of greater toxicity. Additionally, the iron chelating agent(s) can be delivered using a sustained-release system, such as semipermeable matrices of solid polymers containing the therapeutic agent. Various sustained-release materials have been established and are well known by those skilled in the art. Sustained-release capsules may, depending on their chemical nature, can release the iron chelating agent(s) for a few days, a few weeks, or up to over 100 days. Depending on the chemical nature and the biological stability of the iron chelating agent(s) additional strategies for stabilization can be employed.

As the iron chelating agent(s) may contain charged side chains or termini, they may be included in any of the above-described formulations as the free acids or bases or as pharmaceutically acceptable salts. Pharmaceutically acceptable salts are those salts which substantially retain the biological activity of the free bases and which are prepared by reaction with inorganic acids. Pharmaceutical salts tend to be more soluble in aqueous and other protic solvents than are the corresponding free base forms.

### B) “Genetic” Delivery Methods

As indicated above, molecules encoding one or more iron binding proteins (e.g. hemoglobin, ferridoxin, fragments thereof, etc.) can be delivered and transcribed and/or expressed in target cells (e.g. vascular endothelial cells) using methods of gene therapy. Thus, in certain preferred embodiments, the nucleic acids encoding one or more iron binding proteins, typically operably linked to a promoter (e.g. constitutive, inducible, tissue specific), are cloned into gene therapy vectors that are competent to transfect cells (such as human or other mammalian nerve cells) in vitro and/or in vivo.

Many approaches for introducing nucleic acids into cells in viva, ex viva and in vitro are known. These include lipid or liposome based gene delivery (WO 96/18372; WO 93/24640; Mannino and Gould-Fogerite (1988) BioTechniques 6(7): 682-691; Rose U.S. Pat No. 5,279,833; WO 91/06309; and Felgner et al. (1987) Proc. Natl. Acad. Sci. USA 84: 7413-7414) and replication-defective retroviral vectors harboring a therapeutic polynucleotide sequence as part of the retroviral genome (see, e.g., Miller et al. (1990) Mol. Cell. Biol. 10:4239 (1990); Kolberg (1992) J. NIH Res. 4: 43, and Cornetta et al. (1991) Hum. Gene Ther. 2: 215).

For a review of gene therapy procedures, see, e.g., Anderson, Science (1992) 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Böhm (eds) Springer-Verlag, Heidelberg Germany; and Yu et al., (1994) Gene Therapy, 1:13-26.

Widely used vector systems include, but are not limited to adenovirus, adeno associated virus, and various retroviral expression systems. The use of adenoviral vectors is well known to those of skill and is described in detail, e.g., in WO 96/25507. Particularly preferred adenoviral vectors are described by Wills et al. (1994) Hum. Gene Therap. 5: 1079-1088.

Adeno-associated virus (AAV)-based vectors used to transduce cells with target nucleic acids, e.g., in the in vitro production of nucleic acids and peptides, and in in vivo and ex vivo gene therapy procedures are describe, for example, by West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al.,(1989) J. Virol., 63:03822-3828. Cell lines that can be transformed by rAAV include those described in Lebkowski et al. (1988) Mol. Cell. Biol., 8:3988-3996.

Widely used retroviral vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5): 1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like). Other suitable viral vectors include, but are not limited to herpes virus, lentivirus, and vaccinia virus.

Alone, or in combination with viral vectors, a number of non-viral vectors are also useful for transfecting cells to express proteins that bind free iron. Suitable non-viral vectors include, but are not limited to, plasmids, cosmids, phagemids, liposomes, water-oil emulsions, polethylene imines, biolistic pellets/beads, and dendrimers.

Liposomes were first described in 1965 as a model of cellular membranes and quickly were applied to the delivery of substances to cells. Liposomes entrap DNA by one of two mechanisms which has resulted in their classification as either cationic liposomes or pH-sensitive liposomes. Cationic liposomes are positively charged liposomes which interact with the negatively charged DNA molecules to form a stable complex. Cationic liposomes typically consist of a positively charged lipid and a co-lipid. Commonly used co-lipids include dioleoyl phosphatidylethanolamine (DOPE) or dioleoyl phosphatidylcholine (DOPC). Co-lipids, also called helper lipids, are in most cases required for stabilization of liposome complex. A variety of positively charged lipid formulations are commercially available and many other are under development. Two of the most frequently cited cationic lipids are lipofectamine and lipofectin. Lipofectin is a commercially available cationic lipid first reported by Phil Felgner in 1987 to deliver genes to cells in culture. Lipofectin is a mixture of N-[1-(2,3-dioleyloyx) propyl]-N-N-N-trimethyl ammonia chloride (DOTMA) and DOPE.

DNA and lipofectin or lipofectamine interact spontaneously to form complexes that have a 100% loading efficiency. In other words, essentially all of the DNA is complexed with the lipid, provided enough lipid is available. It is assumed that the negative charge of the DNA molecule interacts with the positively charged groups of the DOTMA. The lipid:DNA ratio and overall lipid concentrations used in forming these complexes are extremely important for efficient gene transfer and vary with application. Lipofectin has been used to deliver linear DNA, plasmid DNA, and RNA to a variety of cells in culture. Shortly after its introduction, it was shown that lipofectin could be used to deliver genes in vivo. Following intravenous administration of lipofectin-DNA complexes, both the lung and liver showed marked affinity for uptake of these complexes and transgene expression. Injection of these complexes into other tissues has had varying results and, for the most part, are much less efficient than lipofectin-mediated gene transfer into either the lung or the liver.

PH-sensitive, or negatively-charged liposomes, entrap DNA rather than complex with it. Since both the DNA and the lipid are similarly charged, repulsion rather than complex formation occurs. Yet, some DNA does manage to get entrapped within the aqueous interior of these liposomes. In some cases, these liposomes are destabilized by low pH and hence the term pH- sensitive. To date, cationic liposomes have been much more efficient at gene delivery both in vivo and in vitro than pH-sensitive liposomes. pH-sensitive liposomes have the potential to be much more efficient at in vivo DNA delivery than their cationic counterparts and should be able to do so with reduced toxicity and interference from serum protein.

In another approach dendrimers complexed to the DNA have been used to transfect cells. Such dendrimers include, but are not limited to, “starburst” dendrimers and various dendrimer polycations.

Dendrimer polycations are three dimensional, highly ordered oligomeric and/or polymeric compounds typically formed on a core molecule or designated initiator by reiterative reaction sequences adding the oligomers and/or polymers and providing an outer surface that is positively changed. These dendrimers may be prepared as disclosed in PCT/US83/02052, and U.S. Pat. Nos. 4,507,466, 4,558,120, 4,568,737, 4,587,329, 4,631,337, 4,694,064, 4,713,975, 4,737,550, 4,871,779, 4,857,599.

Typically, the dendrimer polycations comprise a core molecule upon which polymers are added. The polymers may be oligomers or polymers which comprise terminal groups capable of acquiring a positive charge. Suitable core molecules comprise at least two reactive residues which can be utilized for the binding of the core molecule to the oligomers and/or polymers. Examples of the reactive residues are hydroxyl, ester, amino, imino, imido, halide, carboxyl, carboxyhalide maleimide, dithiopyridyl, and sulfhydryl, among others. Preferred core molecules are ammonia, tris-(2-aminoethyl)amine, lysine, ornithine, pentaerythritol and ethylenediamine, among others. Combinations of these residues are also suitable as are other reactive residues.

Oligomers and polymers suitable for the preparation of the dendrimer polycations of the invention are pharmaceutically-acceptable oligomers and/or polymers that are well accepted in the body. Examples of these are polyamidoamines derived from the reaction of an alkyl ester of an α,β-ethylenically unsaturated carboxylic acid or an α,β-ethylenically unsaturated amide and an alkylene polyamine or a polyalkylene polyamine, among others. Preferred are methyl acrylate and ethylenediamine. The polymer is preferably covalently bound to the core molecule.

The terminal groups that may be attached to the oligomers and/or polymers should be capable of acquiring a positive charge. Examples of these are azoles and primary, secondary, tertiary and quaternary aliphatic and aromatic amines and azoles, which may be substituted with S or O, guanidinium, and combinations thereof. The terminal cationic groups are preferably attached in a covalent manner to the oligomers and/or polymers. Preferred terminal cationic groups are amines and guanidinium. However, others may also be utilized. The terminal cationic groups may be present in a proportion of about 10 to 100% of all terminal groups of the oligomer and/or polymer, and more preferably about 50 to 100%.

The dendrimer polycation may also comprise 0 to about 90% terminal reactive residues other than the cationic groups. Suitable terminal reactive residues other than the terminal cationic groups are hydroxyl, cyano, carboxyl, sulfhydryl, amide and thioether, among others, and combinations thereof. However others may also be utilized.

The dendrimer polycation is generally and preferably non-covalently associated with the polynucleotide. This permits an easy disassociation or disassembling of the composition once it is delivered into the cell. Typical dendrimer polycation suitable for use herein have a molecular weight ranging from about 2,000 to 1,000,000 Da, and more preferably about 5,000 to 500,000 Da. However, other molecule weights are also suitable. Preferred dendrimer polycations have a hydrodynamic radius of about 11 to 60 Å., and more preferably about 15 to 55 Å. Other sizes, however, are also suitable. Methods for the preparation and use of dendrimers in gene therapy are well known to those of skill in the art and describe in detail, for example, in U.S. Pat. No. 5,661,025.

Where appropriate, two or more types of vectors can be used together. For example, a plasmid vector may be used in conjunction with liposomes. In the case of non-viral vectors, nucleic acid may be incorporated into the non-viral vectors by any suitable means known in the art. For plasmids, this typically involves ligating the construct into a suitable restriction site. For vectors such as liposomes, water-oil emulsions, polyethylene amines and dendrimers, the vector and construct may be associated by mixing under suitable conditions known in the art.

### C) Effective Dosages

The iron chelating agents, iron binding proteins, and the like will generally be used in an amount effective to achieve the intended purpose (e.g. to reduce or prevent onset or progression of a disease characterized by neruological degereration). In preferred embodiments, iron chelating agents, iron binding proteins, and the like utilized in the methods of this invention are administered at a dose that is effective to partially or fully inhibit the onset or progression of one or more symptoms of a disease characterized by neurological degeneration (e.g. Parkinson's disease) (e.g., in certain embodiments, a statistically significant decrease at the 90%, more preferably at the 95%, and most preferably at the 98% or 99% confidence level). Preferred effective amounts are those that reduce or prevent neurological degeneration or improve recovery from neurological degeneration. The compounds can also be used prophalactically at the same dose levels.

Typically, the iron chelating agents, iron binding proteins, and the like, or pharmaceutical compositions thereof, are administered or applied in a therapeutically effective amount. A therapeutically effective amount is an amount effective to reduce or prevent the onset or progression of one or more symptoms of a disease characterized by neurological degeneration. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One skilled in the art could readily optimize administration to humans based on animal data.

Dosage amount and interval may be adjusted individually to provide plasma levels of the inhibitors which are sufficient to maintain therapeutic effect.

Dosages for typical therapeutics, particularly for iron chelating agent(s), are known to those of skill in the art. Moreover, such dosages are typically advisorial in nature and may be adjusted depending on the particular therapeutic context, patient tolerance, etc. Single or multiple administrations of the compositions may be administered depending on the dosage and frequency as required and tolerated by the patient.

In certain embodiments, an initial dosage of about 1μ, preferably from about 1 mg to about 1000 mg per kilogram daily will be effective. A daily dose range of about 5 to about 75 mg is preferred. The dosages, however, may be varied depending upon the requirements of the patient, the severity of the condition being treated, and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstance is reached. F or convenience, the total daily dosage may be divided and administered in portions during the day if desired. Typical dosages will be from about 0.1 to about 500 mg/kg, and ideally about 25 to about 250 mg/kg.

In cases of local administration or selective uptake, the effective local concentration of the inhibitors may not be related to plasma concentration. One skilled in the art will be able to optimize therapeutically effective local dosages without undue experimentation. The amount of inhibitor administered will, of course, be dependent on the subject being treated, on the subject's weight, the severity of the affliction, the manner of administration and the judgment of the prescribing physician.

The therapy may be repeated intermittently. The therapy may be provided alone or in combination with other drugs and/or procedures.

### D) Toxicity

Preferably, a therapeutically effective dose of the iron chelating agents, iron binding proteins, and the like described herein will provide therapeutic benefit without causing substantial toxicity.

Toxicity of the inhibitors described herein can be determined by standard pharmaceutical procedures in cell cultures or experimental animals, e.g., by determining the LD₅₀ (the dose lethal to 50% of the population) or the LD₁₀₀ (the dose lethal to 100% of the population). It is noted that toxicity of numerous iron chelating agent(s) is well characterized. The dose ratio between toxic and therapeutic effect is the therapeutic index. Inhibitors which exhibit high therapeutic indices are preferred. Data obtained from cell culture assays and animal studies can be used in formulating a dosage range that is not toxic for use in human. The dosage of the AGENTS described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition (see, e.g., Fingl et al. (1975) In: The Pharmacological Basis of Therapeutics, Ch.1, p.1).

### III. Diagnostic Methods

In certain embodiments this invention provides methods for evaluating the risk or progression of a disease characterized by neural degeneration in a mammal. The methods typically involve providing a biological sample from the mammal; and determining the level of free iron in the sample where an elevated level of free iron as compared to that found in a sample from a normal healthy mammal indicates that the mammal is at risk for or progressing with the disease. Alternatively, it is possible to assay for endogenous agents that sequester/chelate free iron, where an increase in such agents indicates that the organism is at reduced risk for progression of the disease.

Methods of measuring free iron in a mammal are well known to those of skill in the art. It will be appreciated that it is possible to directly measure levels of free iron (e.g. in plasma), or alternatively, to measure levels of bound iron (e.g. ferritin bound iron) to calculate free iron levels. Methods of measuring free iron levels are described in detail by e.g., Pootrakul et al. (1988) Blood, 71(4): 1120-1123, Tietz (ed.) (1986) Pp. 1577-1584, 914-915 In: Textbook of Clinical Chemistry, W. B. Saunders Company, Zuyderhoudt et al. (1978) Clinica Chimica Acta, 86: 313-321, Zuyderhoudt et al. (1978) Clinica Chimica Acta. 90: 93-99, and the like. In addition, biosensors for detecting iron are known to those of skill in the art (see, e.g., U.S. Pat. No. 5,516,697).

### IV. Screening for Agents that Inhibit Neural Degeneration in a Mammal

In certain embodiments, this invention provides methods of screening for agents that inhibit neural degeneration in a mammal. Typically the methods involve screening a test agent for the ability to sequester/chelate free iron, and/or for the ability to induce an organism, tissue, and/or cell to sequester/chelate iron, and/or to upregulate production of endogenous agent(s) that sequester/chelate iron.

In various embodiments, the methods involve contacting an animal, tissue, and/or cell, with one or more test agents and evaluating the effect of the test agent on the sequestration/chelation of free iron. Methods of detecting iron chelation/sequestration are well known to those of skill in the art and are illustrated herein in the Examples.

### V. Kits

In another embodiment, this invention provides kits for practice of the methods of this invention. Such kits preferably include a container containing one or more iron chelating agents and/or nucleic acid constructs encoding iron chelating proteins. The iron chelating agent(s) can be formulated in combination with a pharmaceutically acceptable excipient and/or in a unit dosage form.

The kit can comprise packaging that retains and presents the medicants (e.g., iron chelating agent(s)) at separate respective consecutive locations identified by visibly discernible indicia and the times at which the medicants are to be taken by the patient. In various embodiments, the times can include each day of the week and specified times within each day presented in the form of a chart located on one face of the package wherein the days of the week are presented and the times within each day the medicants are to be taken are presented in systematic fashion.

In addition, the kits can include instructional materials containing directions teaching the use of one or more iron chelating agent(s) or constructs encoding iron binding proteins to reduce/inhibit the onset or progression of a disease characterized by neurological degeneration (e.g. Parkinson's disease). While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Non-Toxic Genetic or Pharmacological Iron Chelation Prevents MPTP-Induced Neurotoxicity In Vivo: A Novel Therapy for Parkinson's Disease

Studies on postmortem brains from Parkinson's patients reveal elevated iron in the substantia nigra (SN). elective cell death in this brain region is associated with oxidative stress which may be exascerbated by the presence of excess iron. hether iron plays a causitive role in cell death, however, is controversial. In this example, we explore the effects of non-toxic iron chelation via either transgenic expression of the iron-binding protein ferritin or oral administration of the bioavailable metal chelator clioquinol (CQ) on susceptibility to the Parkinson's-inducing agent 1-methyl-4-phenyl-1,2,3,6-tetrapyndine (MPTP). Reduction in reactive iron by either genetic or pharmacological means results in protection against the toxin suggesting that non-toxic iron chelation may be an effective therapy for prevention and treatment of the disease.

### Introduction

This example described experiments undertaken to test whether iron is causally involved in cellular degeneration associated with toxin-induced Parkinsonism by assessing whether iron chelation can act to protect against dopaminergic cell loss.

In the first set of experiments, susceptibility of transgenic mice expressing the ferritin heavy subunit (H ferritin) within dopaminergic SN neurons to the PD-inducing neurotoxin 1-methyl-4-phenyl-1,2,3,6-tetrahydropyridine (MPTP) was assessed. Ferritin, the primary non-heme iron storage molecule in the body, can sequester up to 4500 atoms of fenic (Fe³⁺) iron as an oxyhydroxide (Harrison and Arosio (1996) Biochim. Biophys. Acta 1275: 161-203). Ferritin is believed to keep iron in a non-reactive form where it cannot promote redox reactions and therefore could be a key component for protecting tissues against iron-catalyzed oxidative damage (Jellinger (1999) Drugs Aging 14: 115-140). The ferroxidase activity of H ferritin converts harmful labile ferrous iron to less soluble, unreactive ferric iron while the light subunit (L ferritin) stablizes the ferritin-iron complex promoting long-term iron storage (Jellinger (1999) Drugs Aging 14: 115-140; Rucker et al. (1996) J. Biol. Chem. 52: 33352-33357). In the second set of experiments, mice were orally pretreated for 8 weeks with the antibiotic 5-chloro-7-iodo-8-hydroxyquinoline (clioquinol or CQ) and assessed for the ability of the compound to protect against MPTP-induced toxicity.

Another antibiotic compound, minocycline, has previously been demonstrated to protect against MPTP toxicity likely due to its ability to decrease nitric oxide-mediated apoptosis (Du et al. (2001) Proc. Natl. Acad. Sci. USA 98: 14669-14674). CQ's mechanism of action, however, is likely different. It has been shown to chelate both ferrous and ferric iron (Kidani et al. (1974) Jap. Analyst 23, 1375-1378) and to decrease brain iron levels (Yassin, et al. (2000) J. Neurol. Sci. 173: 40-44). Its oral administration was recently reported to inhibit beta-amyloid accumulation in an Alzheimer's disease (AD) transgenic mouse model via its actions as a metal chelator (Huang et al. (1999) J. Biol. Chem. 274: 37111-37116; Bush and Masters (2001) Science 292: 2251-2252; Cherny et al. (2001) Neuron 30: 665-676). Neither ferritin expression nor oral CQ treatment elicited any apparent adverse general health or behavioral effects unlike chelators currently used as therapy for iron overload conditions which can have severe side effects (Porter and Huehns (1989) Baillieres Clin. Haematol. 2: 459-474; Marciani et al. (1991) Haematologica 76: 131-134). Results from our studies demonstrate that non-toxic in vivo iron chelation protects mice against the toxic effects of the Parkinsonian-inducing agent MPTP and suggest that this may be a novel avenue of therapy for the disease.

### Experimental Procedures

### Mouse Studies

Mice were housed according to standard animal care protocols, fed ad libitum, kept on a 12-hr light/dark cycle and maintained in a pathogen-free environment in the Buck Institute Vivarium. Animals used for studies were young adults (2-6 months of age). Ferritin transgenic mice were generated via injection of an 8.3 kb Hind III DNA fragment containing 4.8 kb of 5′ upstream sequences from the rat TH gene (Banjeree et al. (1992) J. Neurosci. 12:4460-4467), 2.6 kb of human genomic ferritin DNA encompassing the 4 coding region exons (Hentze et al., 1986), and 3′ SV40 splice and poly-adenylation sequences into fertilized B6D2 mouse embryos to create pTH-ferretin transgenic founder animals. For CQ studies, C57B1 mice were obtained from Jackson Labs and randomized for therapy trials. CQ was suspended in 0.05% carboxymethylcellulose (Sigma) and delivered via oral gavage at a daily dosage of 30 mg/kg as previously described for a period of 8 weeks (Cherny et al. (2001) Neuron 30: 665-676); controls received vehicle alone.

### Southern Blot Analysis

Genomic DNA from ferritin founders was digested with Xba I, separated on a 1% agarose gel, transferred to Hybond (Amersham) and hybridized with a ³²P-labeled 2.6 kb Xba I-EcoRI ferritin genomic fragment. Founder animals positive for the transgene were bred out to create lines for analysis; non-transgenic littermates were used as negative controls.

### Western/Slot Blot Analyses

SN were dissected and homogenized in 10 mM HEPES-KOH, pH 7.2, 2 mM EDTA, 0.1% CHAPS, 5 mM dithiothreitol (DTT), 1 mM phenylmethylsulfonyl fluoride, 10 μg/ml pepstatin A, 10 μg/ml aprotinin, and 20 μg/ml leupeptin (Nicholson et al. (1995) Nature 376: 37-43). 15 μg of total protein from each sample was either run on a 15% SDS-PAGE gel (BioRad) and transferred to nitrocellulose membrane or directly slotted onto membrane. Membranes were incubated with 10-50 μg/ml primary antibody (heavy chain human ferritin monoclonal, Ramco Laboratories; anti-HNE Michaels adduct rabbit polyclonal, Calbiochem; anti-DNP rabbit polyclonal, Intergen) followed by horseradish peroxidase-conjugated secondary antibody (Vector Laboratories). Autoradiography was performed with enhanced chemiluminescence (Amersham Pharmacia). For 4HNNE-protein conjugates and protein carbonyls, relative optical band density were quantified using a Chemilmager 5500 (Alpha Innotech Corporation). Reported values are the results of three independent experiments.

### Immunocytochemistry

Animals were cardiac-perfused with phosphate buffered saline (PBS) followed by 10% formalin, brains removed and post-fixed for 15 hrs followed by 30% sucrose and sectioning at 40 μm on the coronal plane. ICC was performed as previously described (Andersen et al. (2001) In Science of Aging Knowledge Environment (SAGE KE) Website (AAAS/Science magazine), October inaugural edition). Specific primary antibodies were applied and visualized with fluorescence (Streptavidin-Cy3 for red fluorescence and Streptavidin-Cy2 for green fluorescence, Jackson Immunochemicals).

### SN Iron Levels by Magnetic Resonance Imaging (MRI)

MRI studies were performed using a Bruker AMX500 11.7 tesla MRI system as previously described (Gilissen et al. (1998) Am. J. Primatol. 45: 291-299). Brains were fixed as described above and MRI performed in the coronal plane. Comparisons of SN hypointensity (dark area) on T2-weighted MR samples encompassing SNc, SNr, and red nucleus were performed (IPLab Spectrum, Scientific Image Processing from Scanalytics, Inc.)(Morgan et al., 1998). Intensity was normalized using cortical white matter as control.

### Spectrophotometric Analysis of Bioavailable Ferrous Iron

Levels of ferrous iron available to bind ferrozine were determined in dissected SN tissue spectrophotometrically at 578 nm as previously described (Agrawal et al. (2001) Toxicology 168: 223-230).

### Ferric Iron Histochemistry (Perls)

Coronal sections from brains of adult animals were subjected to formalin fixation and Perls staining using potassium ferrocyanide as previously described (Hill and Switzer (1984) Neurosci. 11: 595-603). The percentage area covered by ferric-ferrocyanine product was assessed by Camera Luminace Drawing.

### ROS by DCF Fluorescence

Animals were i.p. injected with either 30 mg/kg body weight MPTP or saline. Eight hours following injection, synaptosomal fractions were prepared from the SN and used for DCF analysis (Ali et al. (1992) Neurotoxicol,. 13: 637-648). Fluorescence was monitored on a Turner spectrofluorometer with an excitation wavelength of 448 mm and an emission wave length of 525 nm. Protein was normalized by the Bradford method.

### GSH Levels

Following MPTP or saline injection, GSH levels were measured in the SN by the method of Griffith (Griffith (1980) Anal. Biochem. 106; 207-212).

### Histology and Neuron Counts

Neuronal counts were performed on TH+ positive SN neurons using the unbiased dissector method (West (1993) Neurobiol. Aging 14: 275-285). Fixed coronal brain sections (40 μm) were immunostained with TH antibody (1:500 dilution, Chemicon), coverslipped in aqueous medium and TH+ cells counted from a total of 15-20 sections in each field per brain (i.e. every second section) at a magnification of 100× using the optical fractionator approach.

### Striatal Dopamine/DOPAC and MPP+ Levels

Animals were injected with either 15 mg/kg body weight MPTP or saline every 2 hrs, 4 doses. Dopamine, DOPAC, and HVA or MPP+ from dissected striata were analyzed by I-PLC using a 5 micrometer C-18 reverse phase column and precolumn (Brownlee Labs) followed by electrochemical detection with a glassy carbon electrode (Klivenyi et al. (2000) J. Neurosci. 20: 1-7).

### SN Iron Levels by Mass Spectrometry

SN was dissected, snap frozen in liquid nitrogen, lypholized and dry weight/tissue measured. Preweighed lypholized samples were next taken up in 0.1 ml of concentrated nitric acid (Aristar, BDH) and allowed to digest overnight. The samples were then heated to 80° C. for 15 min, cooled and 0.1 ml 30% hydrogen peroxide added. Samples were heated to 70° C. for 15 min, cooled, and diluted 1/40 into 1% HN03 for analysis by inductively coupled plasma mass spectrometry (ICP-MS) using an Ultramass 700 (Varian) in peak-hopping mode with 0.100 AMU spacing, 1 point per peak, 50 scans per replicate, 3 replicates per sample. Preparation blanks processed in a similar manner were used as controls. Plasma flow was 15 L/min with auxiliary flow of 1.5 L/min, RF power was 1.2 kW, and sample was introduced at a flow rate of 0.88 L/min.

### MAO-B Activity

Brain homogenates were analyzed by the toulene extraction method using 10 μM 14-C labeled PEA as substrate (NEN, 56 mCi/mol) as previously described (Wei et al. (1996) J. Neurosci. Res. 46: 666-673; Wei et al. (1997) J. Neurosci. Res. 50: 618-626). Values are reported as cpm/μg protein.

### Open Field Analysis of Motor Activity in Ferritin Transgenics and CO-Fed Animals vs. Controls

Ferritin transgenics vs. wild type littermates and CQ-fed vs. saline-fed animals used for open field behavioral studies were 2-5 months of age. 6-8 mice in each category were tested. Behavioral parameters were monitored in a Coulbourn Instrument Tru Scan activity monitor. Motor activities were recorded every 100 ms for a 10 minute period. Tru Scan 99 software was used to generate experimental protocols and to acquire and store the data. The data was exported to EXCEL and analyzed via one way ANOVA.

### Results

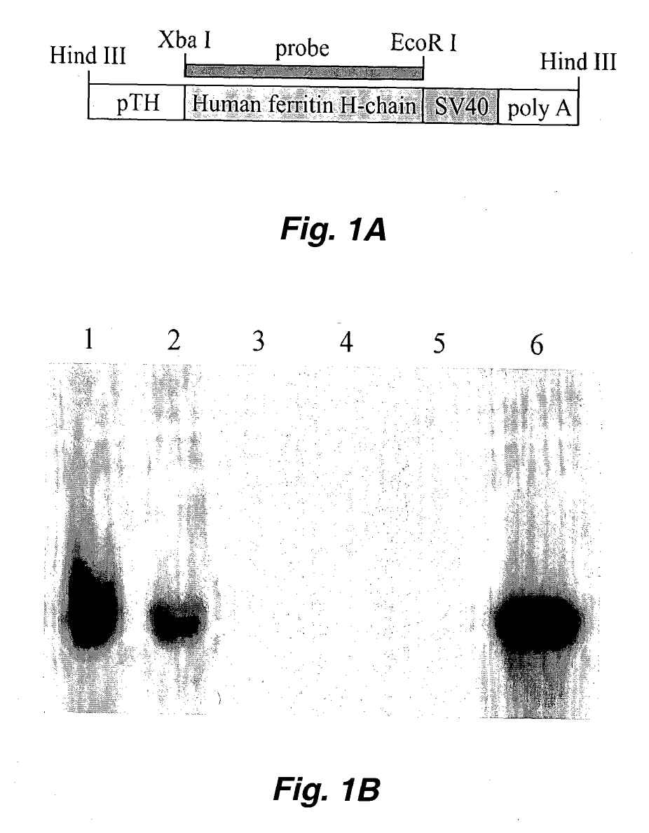

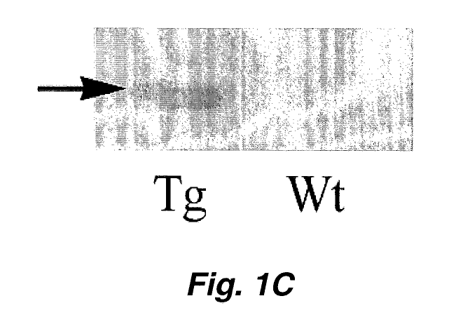

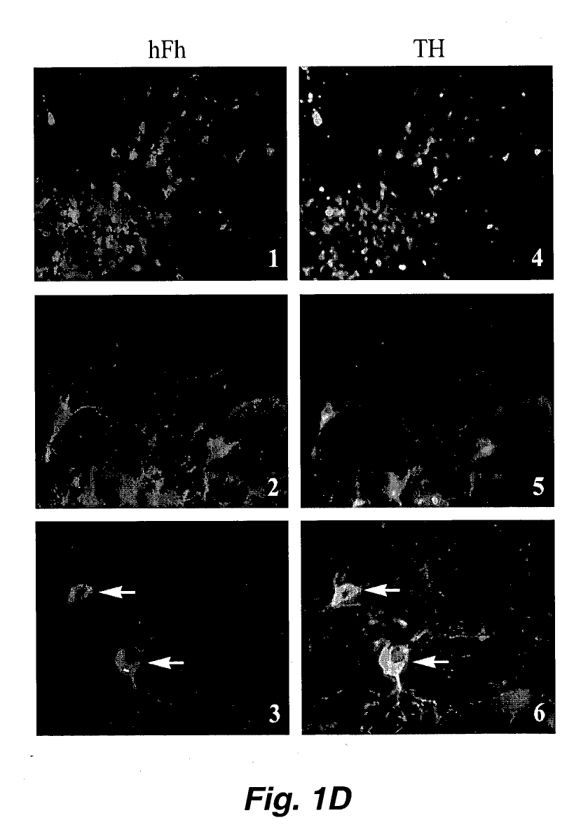

Transgenic ferritin lines were generated by injection of an 8.3 kb DNA fragment into fertilized mouse embryos containing the rat tyrosine hydroxylase promoter (pTH) driving expression of the human H-ferritin gene (FIG. 1A). Human ferritin binds iron more tightly than the mouse isoform making it a superior iron chelating agent and monoclonal antibodies are also available which are specific to the human protein (Rucker et al., 1996). In order to prevent irdn-induced down-regulation of transgenic ferritin RNA translation, the 5′ non-coding region of the gene containing an iron-response element (IRE) was excluded from the construct (Caughman et al. (1988) J. Biol. Chem. 263: 19048-19052).

Integration of the pTH-ferritin transgene in founder animals was verified by Southern blot analysis (FIG. 1B). Expression of the human H-ferritin protein in the SN of resulting lines was verified by both Western blot analysis (FIG. 1C) and immunocytochemistry (ICC, FIGS. 1D, 1-3). No changes were observed in endogenous ferritin levels in these animals (data not shown). Double labeling of H ferritin-expressing cells with tyrosine hydroxylase (TH) antibody demonstrated that the transgenic ferritin protein is localized within dopaminergic SN neurons (FIGS. 1D, 4-6).

Adult ferritin transgenics exhibited no overt phenotype, reproduced normally, and displayed no gross alterations in brain size or anatomical features in histologically-stained brain sections (data not shown). To assess the effects of SN H-ferritin expression on behavior in more detail, an open field exam using a broad battery of spontaneous motor activity measurements was performed (Table 1). An automated photochamber surveillance system was used to remove observer bias (Weiss (1999) Pp. 649-673. In General and Applied Toxicology, Ballantyne, B., Marrs, T. C., and Syversen, T., eds. (Macmillian)). No adverse behavioral effects were observed in ferritin animals in 12 separate motor activity assays including indices of locomoter behavior, circling, rearing, and stereotypic behavior (n=6-8 animals per analysis, p<0.01).

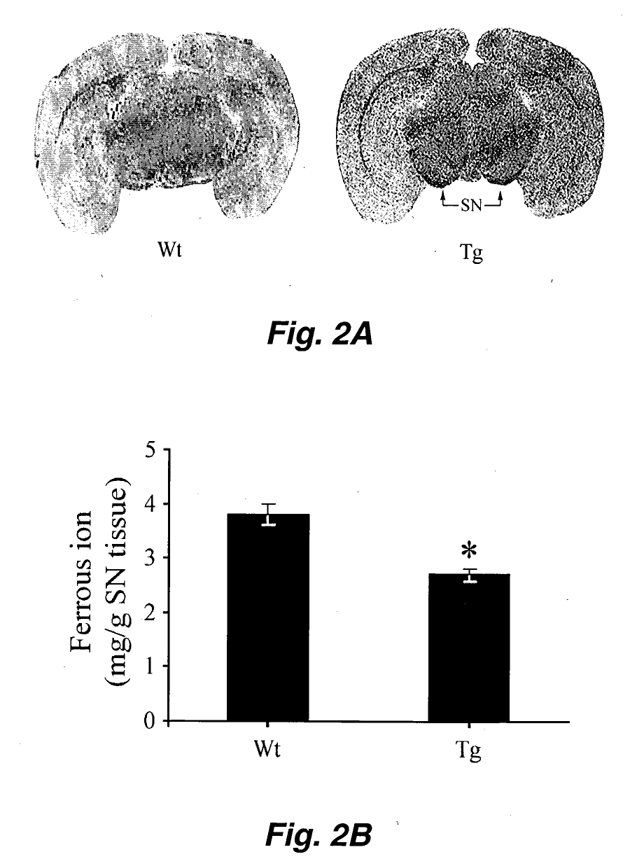

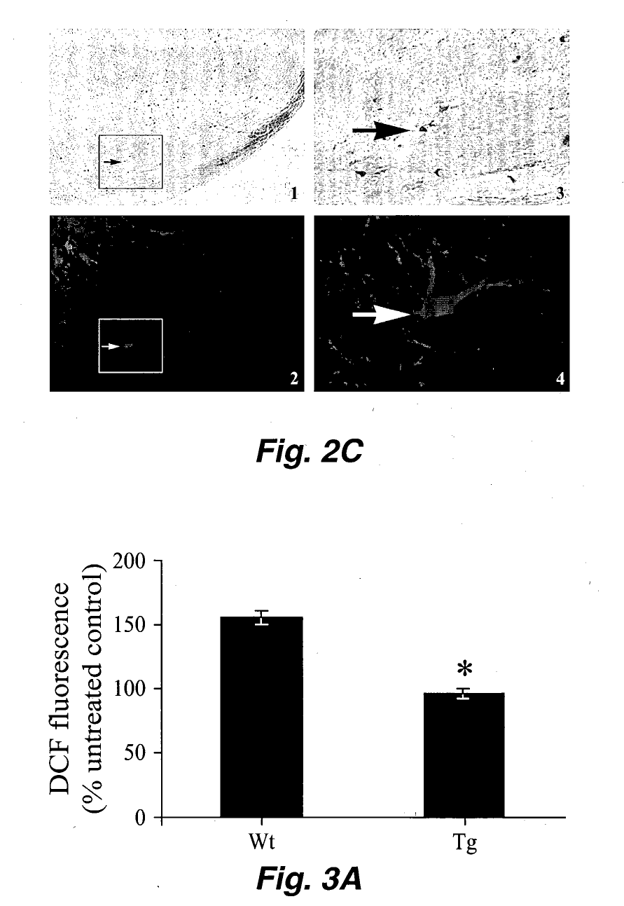

Increased iron binding to ferritin in would be expected to result in increased conversion of ferrous to ferric iron as it enters the ferritin core and is oxidized to ferrihydrite. Ferric iron's paramagnetic characteristics allow for its visualization by high field magnetic resonance imaging (MRI); the signal is intensified when iron is bound to ferritin and thereby can be used as a measure of ferritin-bound iron (Gilissen et al. (1998) Am. J. Primatol. 45: 291-299; Griffiths et al. (1999) Brain 122: 667-673). MRI was performed on brains from ferritin transgenics vs. non-transgenic litterimates and the signal intensity quantified using frontal cortex as an internal control. A 33.7%±8.5 increase in signal intensity was observed in transgenic animals vs. wild-type controls (FIG. 2A, n=4 animals per parameter, p <0.01). Conversely, bioavailable SN ferrous iron levels were found to be decreased by 22%±9.8 in the ferritin transgenic SN (FIG. 2B, Wt=3.8±0.20 μg/g SN tissue, Tg=2.7±0.13 μg/g SN tissue, n=4 animals per parameter, p<0.01) presumably due to its conversion to ferric during oxidation and storage in ferritin. To assess whether the increased ferric iron co-localized with dopaminergic SN neurons, Perls staining was performed in conjunction with immunocytochemistry using an antibody specific for TH (FIG. 2C). Perls staining revealed that, in agreement with previous reports (Benkovic and Connor (1993) J. Comp. Neurol. 338: 97-113; Connor et al. (1994) J. Neurosci. Res. 37: 461-465; Cheepsunthorn et al. (1998) J. Comp. Neurol. 400: 73-86), ferric iron is predominantly localized within SN cells with the appearance of oligodendrocytes in wildtype animals (data not shown). The numbers of ferric iron-positive cells were increased in the transgenic SN and were found to be localized within cell bodies and neuritic processes of TH-positive SN cells (FIG. 2C). Estimations of numbers of Perls-positive SN cells demonstrated a 22.4%±4.7 increase in the transgenic animals (p<0.01); these cells displayed the correct size, morphology, and TH-positive expression of dopaminergic neurons.

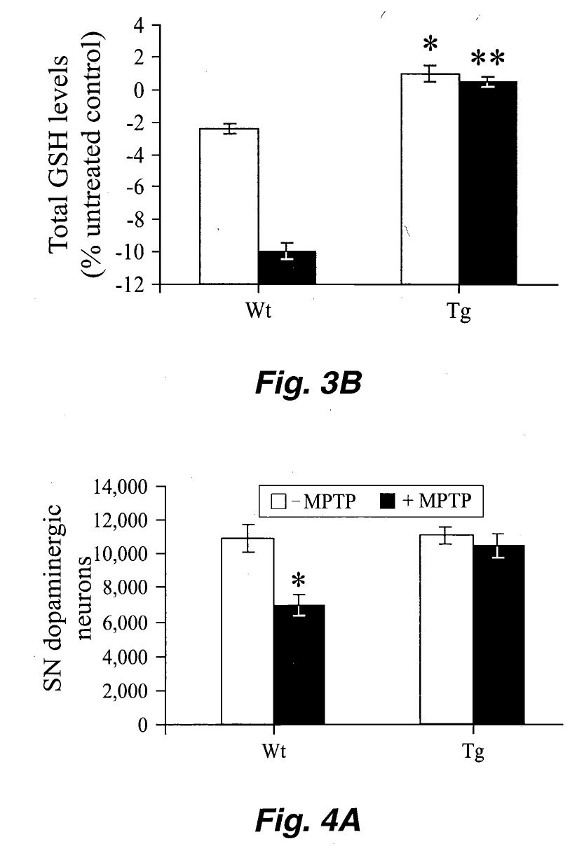

Systemic administration of the neurotoxin MPTP produces a clinical syndrome strikingly similar to PD (Tetrud and Langston (1989) Acta. Neuro. Scand. Suppl 126: 35-40; Chiueh and Rauhala (1998) Ad. Pharm. 42: 796-800). Animals treated with MPTP exhibit several of the major hallmarks of PD including a substantial decrease in numbers of dopaminergic SN neurons. The damaging effect of MPTP administration also mirrors the disease in that oxidative stress appears to play a major role in ensuing neurodegeneration (Yong et al. (1986) Neurosci. Lett. 63: 56-60; Cassarino et al. (1999) Biochem. Biophys. Acta 1453: 49-62) including a decrease in glutathione (GSH) levels as has been reported to occur early in the course of PD (Perry et al. (1982) Neurosci. Lett. 33: 305-310; Sian et al. (1994) Ann. Neurol 36: 348-355; Hung and Lee (1998) Free Rad. Biol. Med 24: 76-84. Desole et al. (1993) Neurosci. Lett. 161: 121-123; Lan and Jiang (1997) J. Neural Transm. 104: 649-660). Both MPTP-induced increases in reactive oxygen species (ROS) and decreases in GSH levels were both found to be prevented in the ferritin transgenics (FIG. 3). MPTP administration (30 mg/kg i.p.) resulted in a 56%±4 increase in ROS levels in wild-type SN (n=6 animals, p<0.01) while no significant change was detected in transgenic animals (95±4.0%, n=8 animals, p<0.05). A 2.4±0.3% decrease in SN GSH was observed at 2 hrs and a 10.0±0.05% at 8 hrs, respectively, after MPTP injection of wild-type mice (n=6), while no significant change was observed in the ferritin transgenics (+1.0±0.5% at 2 hrs and +0.5±0.3% at 8 hrs, respectively, n=8, p<0.05).

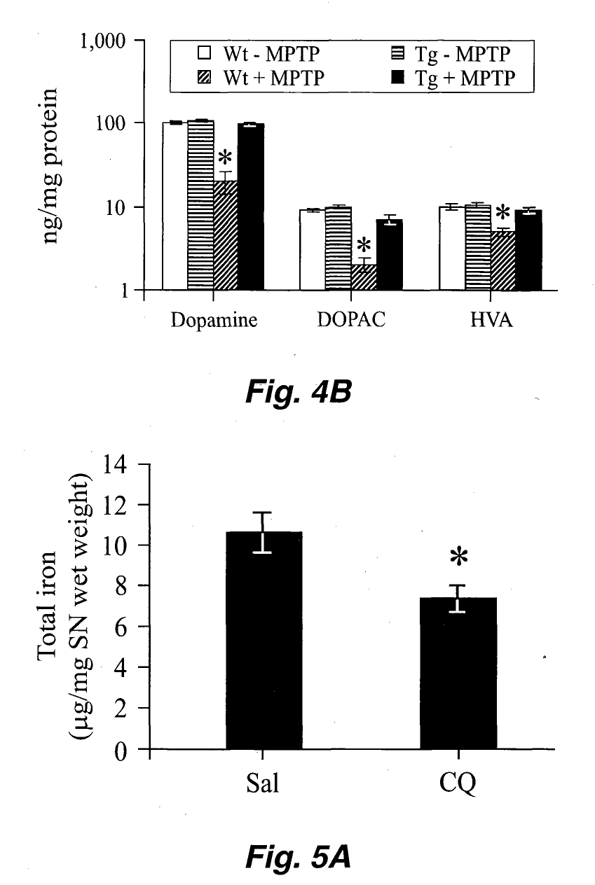

To assess the affects of ferritin expression on MPTP-induced dopaminergic SN cell loss, stereological TH⁺ cell counts were performed. TH⁺ cell numbers in the non-transgenic SN were found to decrease by approximately 30±5.2% following MPTP administration (FIG. 4A, 10,900±800, saline-treated; 7,000±500, MPTP-treated, n=5, p>0.001). In contrast, no decrease was noted following MPTP administration in the ferritin transgenics (11,100±600, saline-treated; 10,500±700, MPTP-treated, n=7, p<0.05). To confirm the protection against TH⁺ SN cell loss in the ferritin transgenic following MPTP administration, levels of striatal dopamine (DA) and its metabolites 3,4-dihydroxyphenylacetic acid (DOPAC) and homovanillic acid (HVA) were measured. Wild-type animals displayed significant depletion of DA, DOPAC, and HVA commensurate with decreased numbers of TH⁺ SN neurons (FIGS. 4B and 4C, DA, DOPAC, IVA=100±3.0, 9.0±0.5, 10.0±0.8 ng/mg protein in saline-treated and 20.0±0.6, 2.0±0.4, 5.0±0.6 ng/mg protein in MPTP-treated, n=4, p<0.001). These losses were attenuated in the ferritin transgenics (DA, DOPAC, HVA=105±3.0, 10.0±0.54, 10.5±0.6 ng/mg protein in saline-treated and 95.0±0.8, 7.0±01.0, 9.0±0.7 ng/mg protein in MPTP-treated, n=5, p>0.05). The protective effects of the ferritin transgene could not be explained by decreased conversion of MPTP to MPP⁺ (transgenic=143.04±18.02 ng/protein MPP⁺, wildtype=109.77±22.03 ng/protein MPP⁺, n=4).

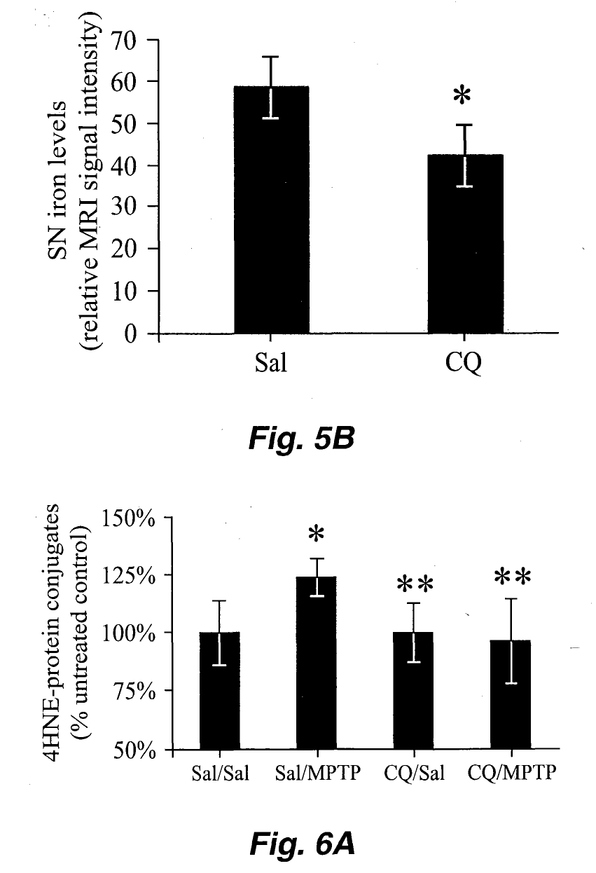

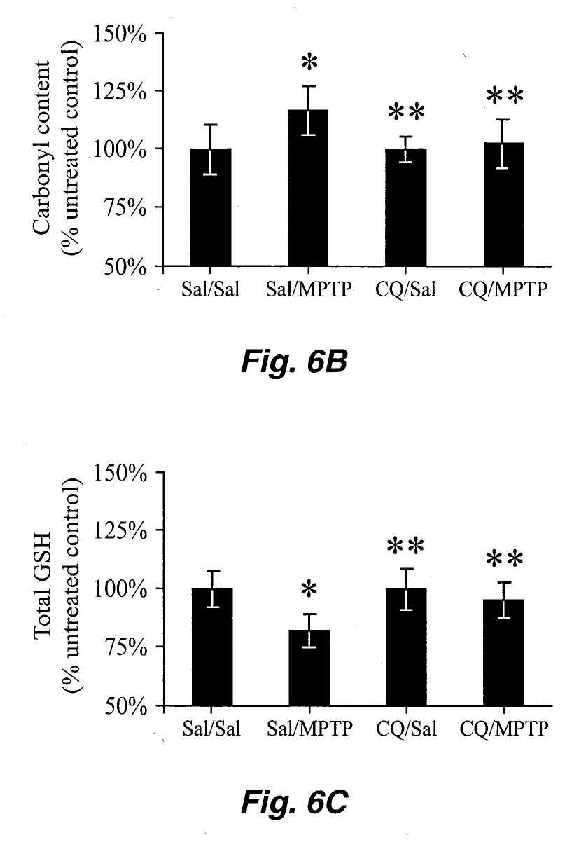

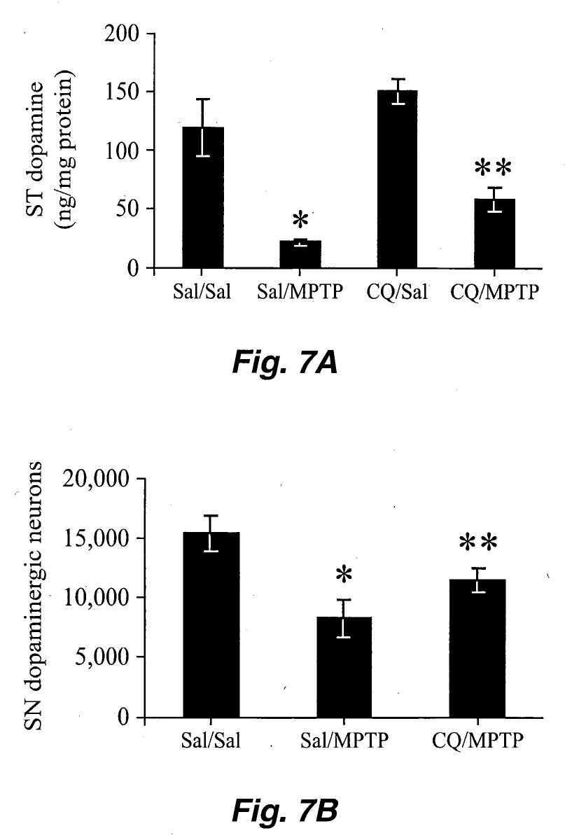

To test whether pharmacological iron chelation using a non-toxic, bioavailable reagent would have similar protective effects afforded by transgenic expression of a iron chelating molecule, we examined the effects of the metal chelating agent CQ on susceptibility to MPTP. Total SN iron levels were found to be reduced approximately 30% in the CQ-fed vs. saline-fed animals (FIG. 5) well within the reported non-toxic range (Yassin, et al. (2000) J. Neurol. Sci. 173: 40-44). As with the ferritin transgenics, MPTP-mediated increases in SN oxidative stress and decreases in SN GSH were found to be significantly attenuated following CQ pretreatment (FIG. 6). A 20%±3 increase in levels of 4-hydroxynonenol (4-HNE)-protein conjugates, a 15±2% increase in protein carbonyl levels, and a 18±3% decrease in GSH were observed in control SN 24 hrs following MPTP injection (n=5 animals per assay, p<0.01). However, no significant changes in any of these indices of oxidative stress were found in the CQ pretreated animals (n=6 animals, p>0.05). To assess the affects of CQ-pretreatment on MPTP-induced dopaminergic SN cell loss, measurements of both striatal dopamine levels and stereological TH⁺ cell numbers were performed (FIG. 7). While reductions in striatal doparmine levels in untreated controls were approximately 80% (121.8±25.6 vs. 21.4±4.5 mg/g striatal tissue), this loss was only 41% in animals pretreated with CQ (147.2±10.6 vs. 60.5±8.8 mg/g striatal tissue). No significant difference in striatal dopamine levels was observed between saline vs. CQ-fed animals in the absence of MPTP treatment suggesting that CQ alone has no effect. While SN TH⁺ cell numbers in untreated animals were decreased by 46% following MPTP administration (15,346±1471, saline-treated; 8,336±1093, MPTP-treated, n=5, p>0.01) only a 25% decrease was noted in the CQ-fed animals (11,462±915, n=5, p>0.01 vs. MPTP-treated controls). The protective effects of CQ pretreatment was not explainable by decreased conversion of MPTP to MPP⁺ as monoamine oxidase-B levels were unchanged by CQ feeding (4000±890 cpm/μg protein, untreated vs. 4500±950 cpm/μg protein, CQ-fed, n=4, p>0.05).

A battery of open field tests examining spontaneous motor activity was also performed on CQ-fed vs. wildtype animals as an indication of motor dysfunction to assess neurotoxicity of the compound. As with the ferritin transgenics, no significant signs of adverse behavioral effects were observed in mice fed CQ up to an 8 week period vs. saline-fed animals (Table 2, n=8 per parameter, p<0.01). This is in keeping with previously published studies showing lack of toxicity of the compound in mice (Cherny et al. (2001) Neuron 30: 665-676) and in recently completed human phase II trials at the same dosages used in these studies.

### Discussion

MPTP-induced neurotoxicity has proven in the past to be an invaluable tool for testing drug therapy in experimental parkinsonism as a model for PD (Sedelis et al. (2001) Behav. Brain Res. 125: 109-125; Beal (2001) Nat. Rev. Neurosci. 2: 325-334). PTP reproduces vitually all symptoms of the disease including inhibition of mitochondrial complex I activity, decreased GSH and increased oxidative stress levels in the SN, relatively selective neurodegeneration of the dopaminergic nigrostriatal system, striatal dopamine depletion, and motor control deficits all of which can be reversed by dopamine substitution therapy, the classic PD drug treatment. Its effects were originally discovered in humans as a consequence of inadvertent injection which resulted in an acute parkinsonism. MPTP does not perfectly model the disorder particularly in terms of the acute nature of onset using this drug and the absence of inclusion bodies in rodents (Betarbet et al. (2002) Bioessays 24: 308-318). An animal model, however, does not need to recapitulate every feature of the disease in order to be useful in evaluating the potential therapeutic potential of a particular agent.

Elevated levels of brain iron similar to those reported in PD have been shown to result in significantly higher levels of both oxidative stress and dopaminergic cell loss following MPTP administration in vivo suggesting that elevated iron can contribute to the toxicity of the compound via an oxidative mechanism (Lan and Jiang (1997) J. Neural Transm. 104: 649-660). Redox available iron has-been detected in midbrain Lewy bodies in post-mortem Parkinsonian brains (Castellani et al. (2000) Proc. Natl. Acad. Sci. USA 98: 14669-14674) and the oxidation state of iron has been reported to change from ferrous to ferric within SN TH⁺ neurons during progression of the disease (Yoshida et al. (2001J. Synchrotron Radiat. 8: 998-1000). Our data demonstrate that chelation of iron via ferritin or CQ in a state that prevents it from participating in oxidative events drastically attenuates toxicity of the compound. These results definitively demonstrate the involvement of iron in MPTP-mediated neurodegeneration as well as addressing its mechanism of action. These results in addition challenge the view that iron accumulation is a late-stage, irreversible event in MPTP toxicity and PD and suggests that iron chelation can be an effective preventative therapy for progressive degeneration associated with the disease.

Transgenic expression of the heavy ferritin subunit was found to prevent dopaminergic SN cell loss associated with MPTP toxicity. The heavy subunit contains catalytic ferroxidase activity which allows it to detoxify reactive ferrous iron and is the predominant form found in brain neurons (Harrison and Arosio (1996) Biochim. Biophys. Acta 1275: 161-203; Connor et al. (1995) J. Neurochem. 65: 717-724; Han et al. (2000) Cell Mol. Biol. 46: 517-528). It is rapidly up-regulated in response to oxidative stress and overexpression in vitro results in increased resistance to H₂0₂-mediated insult (Orino et al. (2001) Biochem. J. 357: 241-247; Cozzi et al. (2000) J. Biol. Chem. 275: 25122-25129) suggesting that it may play an important role as a biological antioxidant by sequestering iron that is normally free to participate in oxidative events. Several recent reports have suggested that diseases of iron overload may have their basis in misregulation of iron storage by ferritin. A dominantly inherited iron overload disease in a Japanese pedigree, for example, was recently attributed to a point mutation in the iron response element (IRE) in the H ferritin gene promoter which leads to increased binding affinity of the iron regulatory protein (IRP), decreasing H ferritin synthesis and resulting in increased cytoplasmic iron levels (Kato, et al. (2001) Am. J. Hum. Genet. 69: 191-197). A mutation in the gene encoding the ferritin light subunit has also recently been reported to cause a dominantly inherited adult-onset basal ganglia disease similar to PD due to a change in its conformation which affects its ability to function as a stabilizer of the ferritin-iron core resulting in increased iron release suggesting that iron excess can have serious neurological consequences (Curtis et al. (2001) Nat. Genet. 28: 350-354; Connor et al. (2001) Pediatr. Neurol. 25: 118-129; Thompson et al. (2001) Brain Res. Bull. 55: 155-164).

Like ferritin, CQ also has metal-binding properties although it appears to act via chelation of both ferrous and ferric iron rather than conversion of available ferrous to bound unreactive ferric iron. It is lipophilic and therefore freely crosses the blood-brain barrier. CQ has recently been shown to inhibit plaque formation and accompanying behavioral declines in an AD transgenic mouse model (Cherny et al. (2001) Neuron 30: 665-676, see commentary by Melov (2002) Trends Neurosci. 25: 121-123). We found that CQ given at similiar concentrations and time periods found to be effective in the AD mouse studies results in significant attenuation of the neurotoxic effects of MPTP. CQ has been shown to reduce bioavailable brain iron in normal control mice (this current study, Yassin, et al. (2000) J. Neurol. Sci. 173: 40-44) with no apparent adverse health or behavioral effects (current study, Cherny et al. (2001) Neuron 30: 665-676). CQ treatment also does not result in depletion in systemic iron levels which could cause adverse physiological effects (Yassin, et al. (2000) J. Neurol. Sci. 173: 40-44). This is in contrast to other currently used iron chelators administered to patients with iron overload conditions which have been shown to have toxic side effects at the higher dosages needed to overcome the compounds' low lipid solubility which impairs their ability to cross the blood-brain barrier (Porter and Huehns (1989) Baillieres Clin. Haematol. 2: 459-474; Marciani et al. (1991) Haematologica 76: 131-134). It should be noted that it cannot be excluded that the protective effects of CQ can be due, in part, to previously reported CQ-mediated depletions in brain copper levels as copper can act to facilitate Fe²⁺ toxicity (Cherny et al. (2001) Neuron 30: 665-676). Phase II clinical trials assessing the efficacy of CQ as a treatment for Alzheimer's disease have recently been completed. No adverse effects were attributable to CQ administration at a similar dosage (g/kg body weight) to that used in the current study when administered in conjunction with B12 supplementation. The results of the phase II trial suggest that CQ accompanied by B12 supplementation is safe in humans despite speculation of its association with a subacute myelo-optic neuropathy (SMON) which was primarily confined to Japan (Tsubaki et al. (1971) Lancet 1: 696-697). CQ has been shown to lower levels of brain and serum vitamin B12 (Yassin, et al. (2000) J. Neurol. Sci. 173: 40-44) and although SMON appears to resemble a subacute accelerated form of B12 deficiency, a causal relationship between SMON and CQ intake has not been established (Meade (1975) Br. J. Prev. Soc Med. 29: 157-169; Nakae et al. (1973) Lancet 1: 171-173; Baumgartner et al. (1979) J. Neurol. Neurosurg. Psychiatry 42: 1073-1083; Clifford Rose and Gawel (1984) Acta Neurol. Scand. Suppl. 100: 137-45). CQ was used extensively in Japan for 20 years before the first cases of SMON were reported and before it was withdrawal from the market, it had been used for over 500 million patient days as an antibiotic with a very favorable safety profile. It has been speculated that the Japanese may have been endemically B12 deficient as a consequence of their diet in the postwar years and that this was a predisposing factor for SMON (Bush and Masters (2001) Science 292: 2251-2252). CQ was used to treat gastrointestinal symptoms in Japan in the post-war era in an unregulated manner which in a B12 deficient population might exaggerate incidence of the disease. In light of this possibility, the Alzheimer phase II clinical trials were performed with B12 co-administration and dosages of the drug were kept to a fraction of those antibiotic dosages used previously.

Increases in reactive brain iron are not specific to PD but are also seen in such diverse neurodegenerative disorders as multiple system atrophy, Huntington's disease, Alzheimer's disease, progressive supranuclear palsy, aceruloplasminemia, and Hallervorden-Spatz (Dexter et al. (1991) Brain 114:1953-1975; Connor et al. (1992) J. Neurosci. Res. 31: 327-335; Smith et al. (1997) Proc. Natl Acad. Sci. 94: 9866-9868; Gitlin (1998). Pediatr. Res. 44:271-276; Janetzky et al. (1997) Pp. 407-421 In: Mitochondria and Free Radicals in Neurodegenerative Diseases, F. Beal, N. Howell, and I. Bodis-Wollner, eds. (Wiley-Liss Inc). Misregulation of iron metabolism resulting in iron accumulation therefore may be a general phenomenon contributing to the progression of several neurodegenerative conditions. Brain iron accumulation along with increased ROS production is part of the normal aging process particularly in the basal ganglia and this in itself may contribute to the increased age-related suceptibility in a subset of these diseases (Bartzokis et al. (1997) Magn. Reson. Imaging 15: 29-35; Zecca et al. (2001) J. Neurochem. 76: 1766-1773; Christen (2000) Am. J. Clin Nutr. 71: 621S-629S; Thompson et al. (2001) Brain Res. Bull. 55: 155-164). Brain H ferritin levels are known to increase with age likely as a protective response to increasing iron levels, however this increase does not appear to occur in either PD or AD brains (Connor et al. (1995) J. Neurochem. 65: 717-724; Zecca et al. (2001) J. Neurochem. 76: 1766-1773; Thompson et al. (2001) Brain Res. Bull. 55: 155-164). Although it has been previously speculated that increasing the iron loading of ferritin may increase the risk of free radical damage (Double et al. (1998) J. Neurochem. 70:2492-2499; Griffiths et al. (1999) Brain 122: 667-673) our data in contrast suggests that increased ferritin is in fact neuroprotective. Indeed ferritin has recently been reported to normally be absent in dopaminergic SN neurons and this may in combination with other factors such as elevated iron levels contribute to their susceptibility to oxidative stress (Moos et al. (2000) Cell Mol. Biol. 46: 549-561). It is of interest in this regard that SN levels of ferritin in humans have been reported to actually be decreased in PD patients compared to age-matched controls, although this is somewhat controversial (Reiderer et al. (1989) J. Neurochem. 52: 515-520; Jellinger et al. (1990) J. Neural Transm. Park Dis. Dement. 2: 327-340; Dexter et al. (1991) Brain 114:1953-1975; Jenner et al. (1992) Neurology 42: 2241-2250; Mann et al. (1994) Ann. Neurol. 36: 876-881). Extensive elimination of iron from the brain is not desirable as it is an abundant brain metal essential for several normal metabolic functions including the synthesis and release of dopamine in the SN (Beard et al. (1993) Prog. Food Nutr. Sci. 17: 183-221; Glinka et al. (1996) P. 1-12 In: In Metals and Oxidative Damage in Neurological Disorders, J. R. Connor, ed. (Plenium Publishing Corp.; Connor et al. (2001) Pediatr. Neurol. 25: 118-129; Thompson et al. (2001) Brain Res. Bull. 55: 155-164). In addition, its deficiency during development has been associated with neurobehavioral dysfunction (Connor et al. (1995) J. Neurochem. 65: 717-724). Our data suggests, however, that non-toxic iron chelators such as ferritin or CQ which can remove excess iron without apparent interference with its normal functions in the adult nervous system system may postpone or prevent the progression of such neurological diseases as PD (Gassen and Youdim (1997) Pharmacol. Toxicol. 80:159-166). CQ possesses an established toxicology profile and clinical trials have demonstrated it as being a safe oral therapy for AD along with B12 supplementation as a prophylactic against possible neurological side effects. We propose based on our data that this compound can also have therapeutic utility in another neurodegenerative condition, Parkinson's disease.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of inhibiting neural degeneration in a mammal, said method comprising reducing free iron levels in a neural tissue of said animal in an amount sufficient to inhibit neural degeneration in said neural tissue.

2. The method of claim 1, wherein said free iron levels are reduced by binding or chelating said iron by contacting said iron with an agent that binds or chelates iron.

3. The method of claim 2, wherein said agent is an iron-chelating small organic molecule.

4. The method of claim 3, wherein said iron-chelating molecule is a molecule selected from the group consisting of 5-chloro-7-iodo-8-hydroxyquinoline (clioquinol), deferiprone, desferrioxamine, pseudan, and derivatives thereof.

5. The method of claim 2, wherein said agent is systemically administered to said mammal.

6. The method of claim 2, wherein said agent is locally administered to a nerve tissue in said mammal.

7. The method of claim 9, wherein said agent is locally administered to brain tissue via a cannula.

8. The method of claim 2, wherein said agent is an iron-binding protein.

9. The method of claim 8, wherein said iron-binding protein is ferritin.

10. The method of claim 9, wherein said iron-binding protein is a ferritin heavy subunit (H ferritin).

11. The method of claim 8, wherein said iron-binding protein is recombinantly expressed.

12. The method of claim 8, wherein said iron-binding protein is recombinantly expressed in vivo.

13. The method of claim 8, wherein said iron-binding protein is recombinantly expressed in a nerve cell.

14. The method of claim 1, wherein said mammal is a human.

15. The method of claim 14, wherein said mammal is a human diagnosed as having or at risk for Parkinson's disease.

16. The method of claim 1, wherein said mammal is a non-human mammal.

17. The method of claim 1, wherein said neural tissue is brain tissue.

18. The method of claim 1, wherein said inhibiting neural degeneration comprises reducing dopaminergic cell loss.

19. A method of inhibiting the onset or progression of a disease characterized by neural degeneration in a mammal, said method comprising reducing free iron levels in a neural tissue of a mammal having or at risk for a disease characterized by neural degeneration.

20. The method of claim 19, wherein said disease is Parkinson's disease.

21. The method of claim 19, wherein said free iron levels are reduced by binding or chelating said iron by contacting said iron with an agent that binds or chelates said iron.

22. The method of claim 21, wherein said agent is an iron-chelating small organic molecule.

23. The method of claim 22, wherein said iron-chelating molecule is a molecule selected from the group consisting of clioquinol, deferiprone, desferrioxamine, pseudan, and derivatives thereof.

24. The method of claim 21, wherein said agent is an iron-binding protein.

25. The method of claim 24, wherein said iron-binding protein is ferritin.

26. The method of claim 25, wherein said iron-binding protein is a ferritin heavy subunit (H ferritin).

27. The method of claim 24, wherein said iron-binding protein is recombinantly expressed.

28. The method of claim 24, wherein said iron-binding protein is recombinantly expressed in vivo.

29. The method of claim 24, wherein said iron-binding protein is recombinantly expressed in a nerve cell.

30. The method of claim 19, wherein said mammal is a human.

31. The method of claim 30, wherein said mammal is a human diagnosed as having or at risk for Parkinson's disease.

32. The method of claim 19, wherein said mammal is a non-human mammal.

33. The method of claim 19, wherein said neural tissue is brain tissue.

34. The method of claim 19, wherein said inhibiting neural degeneration comprises reducing dopaminergic cell loss.

35. A method of mitigating one or more symptoms of a disease characterized by neural degeneration in a mammal, said method comprising administering to said mammal an agent that causes the sequestration or chelation of free iron in said mammal in an amount to mitigate one or more symptoms of said disease.

36. The method of claim 35, wherein said disease is Parkinson's disease.

37. The method of claim 35, wherein said administering comprises administering an iron chelator to said mammal.

38. The method of claim 37, wherein said iron-chelator is a molecule selected from the group consisting of clioquinol, deferiprone, desferrioxamine, pseudan, and derivatives thereof.

39. The method of claim 35, wherein said administering comprises administering an agent that upregulates expression of an endogenous iron chelator.

40. The method of claim 38, wherein said agent upregulates endogenous ferritin or hemoglobin.

41. The method of claim 35, wherein said administering comprises recombinantly expressing an iron-binding protein in a cell of said mammal.

42. The method of claim 41, wherein said iron-binding protein is ferritin.

43. The method of claim 41, wherein said iron-binding protein is a ferritin heavy subunit (H ferritin).

44. The method of claim 41, wherein said iron-binding protein is recombinantly expressed in vivo.

45. The method of claim 41, wherein said iron-binding protein is recombinantly expressed in a nerve cell.

46. The method of claim 19, wherein said mammal is a human.

47. The method of claim 19, wherein said mammal is a human diagnosed as having or at risk for Parkinson's disease.

48. The method of claim 19, wherein said mammal is a non-human mammal.

49. The method of claim 19, wherein said neural tissue is brain tissue.

50. The method of claim 19, wherein said inhibiting neural degeneration comprises reducing dopaminergic cell loss.

51. A method of inhibiting the onset or progression of a disease characterized by neural degeneration in a mammal, said method comprising reducing free iron levels in a neural tissue of a mammal having or at risk for a disease characterized by neural degeneration.

52. The method of claim 51, wherein said disease is Parkinson's disease.

53. The method of claim 51, wherein said free iron levels are reduced by binding or chelating said iron by contacting said iron with an agent that binds or chelates said iron.

54. The method of claim 53, wherein said agent is an iron-chelating small organic molecule.

55. The method of claim 54, wherein said iron-chelating molecule is a molecule selected from the group consisting of clioquinol, deferiprone, desferrioxamine, pseudan, and derivatives thereof.

56. The method of claim 53, wherein said agent is an iron-binding protein.

57. The method of claim 56, wherein said iron-binding protein is ferritin.

58. The method of claim 57, wherein said iron-binding protein is a ferritin heavy subunit (H ferritin).

59. The method of claim 56, wherein said iron-binding protein is recombinantly expressed.

60. The method of claim 56, wherein said iron-binding protein is recombinantly expressed in vivo.

61. The method of claim 56, wherein said iron-binding protein is recombinantly expressed in a nerve cell.

62. The method of claim 51, wherein said mammal is a human.

63. The method of claim 51, wherein said mammal is a human diagnosed as having or at risk for Parkinson's disease.

64. The method of claim 51, wherein said mammal is a non-human mammal.

65. The method of claim 51, wherein said neural tissue is brain tissue.

66. The method of claim 51, wherein said inhibiting neural degeneration comprises reducing dopaminergic cell loss.

67. A kit for mitigating the onset or progression of a disease characterized by neural degeneration in a mammal, said kit comprising:
an agent that increases sequestration or chelation of free iron in said mammal; and
instructional materials teaching the sequestration or chelation of free iron to mitigate the onset or progression of said disease.

68. The composition of claim 67, wherein said composition is formulated in a unit dosage formulation for mitigating the onset or progression of a disease characterized by neural degeneration in a human.

69. The composition of claim 67, wherein said disease is Parkinson's disease.

70. The composition of claim 67, wherein said agent comprises a nucleic acid that encodes a protein that chelates iron.

71. The composition of claim 70, wherein said protein is a ferritin.

72. The composition of claim 67, wherein said agent comprises an iron chelator.

73. The composition of claim 78, wherein said iron-chelator is a molecule selected from the group consisting of clioquinol, deferiprone, desferrioxamine, pseudan, and derivatives thereof.

74. A pharmaceutical composition for mitigating the onset or progression of a disease characterized by neural degeneration in a mammal, said composition comprising an agent that increases sequestration or chelation of free iron in said mammal.

75. The composition of claim 74, wherein said composition is formulated in a unit dosage formulation for-mitigating the onset or progression of a disease characterized by neural degeneration in a human.

76. The composition of claim 74, wherein said disease is Parkinson's disease.

77. The composition of claim 74, wherein said agent comprises a nucleic acid that encodes a protein that chelates iron.

78. The composition of claim 77, wherein said protein is a ferritin.

79. In a mammal diagnosed as having or as at risk for a disease characterized by neural degeneration, a neural tissue in contact with an agent that chelates or sequesters free iron.

80. The neural tissue of claim 79, wherein said mammal is not diagnosed as having an iron overload disease.

81. The neural tissue of claim 79, wherein said agent is an iron chelator.

82. The neural tissue of claim 81, wherein said iron-chelator is a molecule selected from the group consisting of clioquinol, deferiprone, desferrioxamine, pseudan, and derivatives thereof.

83. The neural tissue of claim 79, wherein said agent is an-iron-binding protein.

84. The neural tissue of claim 83, wherein said iron-binding protein is ferritin.

85. The neural tissue of claim 83, wherein said iron-binding protein is a ferritin heavy subunit (H ferritin).

86. The neural tissue of claim 83, n said iron-binding protein is recombinantly expressed.

87. The neural tissue of claim 83, wherein said iron-binding protein is recombinantly expressed in vivo.

88. The neural tissue of claim 83, wherein said iron-binding protein is recombinantly expressed in a nerve cell.

89. The neural tissue of claim 79, wherein said mammal is a human.

90. The neural tissue of claim 79, wherein said mammal is a non-human mammal.

91. The neural tissue of claim 79, wherein said neural tissue is brain tissue.

92. A method of evaluating the risk or progression of a disease characterized by neural degeneration in a mammal, said method comprising:
providing a biological sample from said mammal; and
determining the level of free iron in said sample where an elevated level of free iron as compared to that found in a sample from a normal healthy mammal indicates that said mammal is at risk for or progressing with said disease.

93. The method of claim 39, wherein said disease is Parkinson's disease.
